(19) United States
(12) Patent Application Publication
(10) Pub. No.: US 2025/0014326 A1
(43) Pub. Date: Jan. 9, 2025

(54) MULTI-CLASS INTERACTIVE SEGMENTATION GRAPHICAL USER INTERFACE

(71) Applicant: VENTANA MEDICAL SYSTEMS, INC., Tucson, AZ (US)

(72) Inventors: Qinle BA, Tucson, AZ (US); Jim F. Martin, Tucson, AZ (US); Satarupa Mukherjee, Tucson, AZ (US); Xiangxue Wang, Tucson, AZ (US); Mohammadhassan Izady Yazdanabadi, Tucson, AZ (US)

(73) Assignee: VENTANA MEDICAL SYSTEMS, INC., Tucson, AZ (US)

(21) Appl. No.: 18/887,979

(22) Filed: Sep. 17, 2024

Related U.S. Application Data
(63) Continuation of application No. PCT/US2023/015939, filed on Mar. 22, 2023.
(60) Provisional application No. 63/269,833, filed on Mar. 23, 2022.

(51) Int. Cl. G06V 10/94; G06V 10/774; G06V 20/69; G06V 20/70

(52) U.S. Cl. CPC G06V 10/945; G06V 10/774; G06V 20/695; G06V 20/698; G06V 20/70; G06V 2201/03

(57) ABSTRACT

Methods, computer-program products and systems are provided to perform actions including: receiving an image and displaying the image using a graphical user interface; receiving at least one first image annotation provided by a user via the graphical user interface; producing a first segmented image using a deep learning model, wherein the deep learning model uses the digital pathology image and the at least one first image annotation; and displaying the first segmented image using the graphical user interface; receiving at least one second image annotation provided by the user via the graphical user interface; producing a second segmented image using the deep learning model, wherein the deep learning model uses the digital pathology image, the at least one first image annotation, and the at least one second image annotation; and displaying the second segmented image using the graphical user interface.

### CROSS REFERENCE TO RELATED APPLICATIONS

The present application is a continuation of and claims priority to International Patent Application No. PCT/US2023/015939, filed on Mar. 22, 2023, which claims priority to U.S. Provisional Patent Application No. 63,269,833 filed on Mar. 23, 2022. The application is also related to U.S. patent application Ser. No. 18/125,043, filed on Mar. 22, 2023, which is a continuation-in-part of International Patent Application No. PCT/US2023/015939, filed on Mar. 22, 2023, which claims priority to U.S. Provisional Patent Application No. 63,269,833 filed on Mar. 23, 2022. Each of these applications is hereby incorporated by reference in its entirety for all purposes.

### FIELD

The present disclosure relates to digital pathology, and in particular, to techniques for segmenting images.

### BACKGROUND

Digital pathology involves scanning of slides (e.g., histopathology or cytopathology glass slides) to produce digital images. The slides can include biosamples (e.g., tissue slides or bioliquids) that have been stained using one or more stains (i.e., dyes) that selectively bind to particular cellular components or tissue types. The digital images may be subsequently processed by using a digital-pathology image-processing technique and/or by being interpreted by a pathologist. This subsequent processing may be used for a variety of reasons, such as predicting or facilitating a diagnosis of a disease, estimating a degree to which a given therapy has been effective for a given subject, predicting a degree to which a given therapy will be effective for a given subject, and/or facilitating the development of a new treatment (e.g., new active agernt, dosage, composition, treatment schedule, etc.

A traditional approach for analyzing digital pathology images is for a trained human pathologist to examine highly magnified portions (i.e., “fields of view”) of a slide and to manually segment the image to identify one or more portions of interest (e.g., so as to exclude background, artifacts, macrophages, etc.) and to then detect and classify signals within the each portion of the image. The segmentation and classification are typically performed at a magnification of 40×-400×. Therefore, when the standard approach is used, generating digital pathology results is a very labor-intensive, time-intensive, and financially expensive effort.

An alternative approach is to use a machine learning model to process digital pathology images. However, this typically involves training the model using a training data set that includes a large number of manually labeled images. Given the level of precision required for segmentation (which typically includes delineating boundaries of regions at pixel-level accuracy) and the quantity of data required to train models equipped to process digital pathology images, generating annotations for training data can require a time investment of a half a year or more from a highly trained pathologist.

Deep learning models are one type of machine learning models that have an architecture so sophisticated that it can frequently learn complex data signatures. However, as the complexity of the model increases, so does the size of the requisite training data set. Thus, despite the power of deep-learning systems, developing such systems and promoting their broad applications in the clinical field is challenging, especially given the data-collection variation, the signal variation and the privacy consideration that affects clinical data.

Digital pathology is a particularly challenging context for training any model, much less deep-learning models. In digital pathology, the stains that are used are highly variable. Further, multiple biomarker dyes (e.g., three or more dyes) are frequently used, meaning that there are even fewer images available (particularly when considering privacy constraints) that depict samples stained with a particular combination of dyes. Further yet, a model trained with one tissue type is not readily transferable to other tissue types. It is often the case that a deep learning model built for a specific dataset (i.e., a specific image domain) fails to perform well even on a similar or related dataset (another different image domain).

Thus, in digital pathology, a model designed for a specific diagnostic assay is not readily reused for another assay due to performance issues. This is also the case when applying a model to the images from the same slides but scanned from a different scanner. (See Aubreville M, et al. (2021). MItosis DOmain Generalization Challenge. 24th International Conference on Medical Image Computing and Computer Assisted Intervention (MICCAI 2021). Zenodo. https://doi.org/10.5281/zenodo.4573978.) This lack of model generalizability in digital pathology leads to the inevitable continuous investment of developing new models even for related tasks.

In addition, platforms using machine learning models are often built end-to-end and thus remain black boxes for end-users. This can result in poor interpretability and also poor model performance (due to users not being made aware of logic problems or how to best modify a model to address a detected issued. Further, this inflexible implementation can prevent using a model trained in one domain from being used in another domain.

Therefore, it is particularly challenging to process digital pathology images to accurately and efficiently segment regions of interest. Further, even if a model is configured to perform this type of segmentation, existing systems are not configured to receive responsive user input that does not lead to over-fitting or to generating a model's utility to be dimished.

### SUMMARY

In some embodiments, a computer-implemented method is provided that includes: receiving an image and displaying the image using a graphical user interface; receiving at least one first image annotation provided by a user via the graphical user interface; producing a first segmented image using a deep learning model, wherein the deep learning model uses the digital pathology image and the at least one first image annotation; and displaying the first segmented image using the graphical user interface; receiving at least one second image annotation provided by the user via the graphical user interface; producing a second segmented image using the deep learning model, wherein the deep learning model uses the digital pathology image, the at least one first image annotation, and the at least one second image annotation; and displaying the second segmented image using the graphical user interface.

The deep learning model may be a three-class model and the at least one first image annotation may include at least one annotation for each class.

At least one image annotation may have been provided by the user is provided using a mouse click on the displayed image.

The mouse click may comprise a left mouse click associated with a first segmentation class.

The mouse click may comprise a right mouse click associated with a second segmentation class.

The mouse click may comprise a combination mouse click associated with a third segmentation class.

The combination mouse click may comprise a double left mouse click.

The deep learning model may have been trained using a first dataset comprising training images with one or more annotations.

The training images may comprise images from a first image domain.

The training images may further comprise images from a second image domain.

The second image domain may be different than the first image domain, and wherein the first image domain and the second image domain each comprise one of: natural scene images, digital pathology images, Immunohistochemistry images, x-ray images, and Hematoxylin and eosin images.

The one or more annotations may comprise one or more simulated click annotations.

The image may be a whole-slide pathology image.

In some embodiments, a system is provided that includes one or more data processors and a non-transitory computer readable storage medium containing instructions which, when executed on the one or more data processors, cause the one or more data processors to perform part or all of one or more methods disclosed herein.

In some embodiments, a computer-program product is provided that is tangibly embodied in a non-transitory machine-readable storage medium and that includes instructions configured to cause one or more data processors to perform part or all of one or more methods disclosed herein.

Some embodiments of the present disclosure include a system including one or more data processors. In some embodiments, the system includes a non-transitory computer readable storage medium containing instructions which, when executed on the one or more data processors, cause the one or more data processors to perform part or all of one or more methods and/or part or all of one or more processes disclosed herein. Some embodiments of the present disclosure include a computer-program product tangibly embodied in a non-transitory machine-readable storage medium, including instructions configured to cause one or more data processors to perform part or all of one or more methods and/or part or all of one or more processes disclosed herein.

The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention as claimed has been specifically disclosed by embodiments and optional features, modification and variation of the concepts herein disclosed may be resorted to by those skilled in the art, and that such modifications and variations are considered to be within the scope of this invention as defined by the appended claims.

### BRIEF DESCRIPTIONS OF THE DRAWINGS

The patent or application file contains at least one drawing executed in color. Copies of this patent or patent application publication with color drawing(s) will be provided by the Office upon request and payment of the necessary fee.

Aspects and features of the various embodiments will be more apparent by describing examples with reference to the accompanying drawings, in which:

FIG. 1 shows an exemplary network for generating a digital pathology image and detecting and labeling regions within the digital image using a deep-learning model and label GUI.

FIG. 2 depicts an embodiment of the multi-class DL-based interactive segmentation workflow.

FIG. 3 depicts an example of pipeline for model building is depicted according to various embodiments.

FIG. 4 depicts an example workflow of the multi-class deep learning system according to various embodiments.

FIG. 5 depicts an embodiment of applying interactive binary segmentation to multi-class segmentation of a DP image.

FIG. 6 depicts an embodiment of a three-class deep learning model and training.

FIG. 7 depicts an example workflow of providing annotations according to various embodiments.

FIG. 8 depicts an example interactive GUI according to various embodiments.

FIG. 9 depicts a visualization of a radial expansion strategy for whole-slide interactive segmentation according to various embodiments.

FIG. 10 depicts visualizations of interactive segmentation results from four image domains according to various embodiments.

In the appended figures, similar components and/or features can have the same reference label. Further, various components of the same type can be distinguished by following the reference label by a dash and a second label that distinguishes among the similar components. If only the first reference label is used in the specification, the description is applicable to any one of the similar components having the same first reference label irrespective of the second reference label.

### DETAILED DESCRIPTION

While certain embodiments are described, these embodiments are presented by way of example only, and are not intended to limit the scope of protection. The apparatuses, methods, and systems described herein may be embodied in a variety of other forms. Furthermore, various omissions, substitutions, and changes in the form of the example methods and systems described herein may be made without departing from the scope of protection.

### Overview

Various embodiments of the invention relate to training a deep learning model for interactive segmentation digital pathology images to identify different regions within the image. Further, a graphical user interface (GUI) can be availed, which can allow a user to provide an image, provide interactions to facilitate the segmentation, and/or provide (subsequent to the segmentation) region-label updates. Thus, the deep learning model provides nearly real-time segmentation results, and the GUI is configured to allow a human reviewer to very efficiently and quickly identify region labels.

### Training Images and Click Annotations

The deep learning model can be trained using a multi-class training data set that has images and corresponding annotations from one or more domains. The annotations may include segmentation annotations, that identify borders of or areas of depictions of different things. The annotations may (but need not) include—for each indicated segment—a class (or label, used interchangeably herein) for the segment. The annotations may include, or may be based on, click annotations. For example, for a given image, each of multiple points can be identified, where each point is within a segment that depicts a particular type of object, person, or being. For each point, the annotation may indicate to which of multiple classes the corresponding segment is assigned.

The multi-class training data set is multi-class in that it identifies segmentations for multiple types of depictions. For example, for a digital pathology image, the classes may include a stroma region, a tumor region, and a background region. As another example, for a natural scenes image, the classes may include each vehicle, each stoplight, and the background (all other portions of the image). A background class can be defined to include depictions of (a) other objects that are not selected from the original mask, as well as (b) pure background, where no objects are annotated in the original mask.

That said, the multiple classes need not have semantic meaning. For example, in a three-class instance, the classess may correspond to: a first tye of region; a second type of region; and a background region (which may, but need not, include one or more other types of regions). What constitutes the first and second types of regions may be arbitrary. To illustrate, for an image of vehicles at a traffic light, the first type of region may be vehicle, stoplight, person, crosswalk, etc. In a case where an image depicts multiple objects of a given selected type (e.g., multiple vehicles, multiple people, etc.), all such depictions may be considered as being of the same selected type of region.

The number of click annotations that are identified for a given training image may, but need not, be predefined. For example, an implementation may be configured such that each training image is associated with one click annotation per class, three click annotations per class, six click annotations total, ten click annotations total, etc.

In some instances, click annotations are automatically identified for training images. For example, some training images may be associated with a ground-truth mask that indicates (or that can be used to determine) to which label each pixel is to be assigned. Such ground-truth masks may change with different click annotation targets. For example, two out of multiple image regions in an input image can be used as an segmentation target and the corresponding ground-truth mask can be generated to indicate the target image regions and ignore rest of the image regions. The same image can also be paired with click annotations targeting another set of image regions and corresponding ground-truth masks for these specific image regions. For each ground-truth masks specific to a set of click annotations, the image regions can belong to one class or two classes. The number of unique labels in a ground-truth mask may be different from a number of classes for which the deep-learning model is to be trained. When there are more unique labels than classes, a subset of the labels can be selected, where the number of labels in the subset is equal to the number of classes minus one (given that a background class can be used). The selection can be (for example) a random selection, arbitrary selection, or biased towards labels associated with the most pixel assignments. When there are fewer unique labels than classes, the corresponding image can be discarded from the training data set.

Using the click annotations, a ground-truth mask can be generated for each image, which can be used to train the deep-learning model. Using the click annotations, additional input maps can be generated for each image, which can be used to train the deep-learning model. The input map (which may be the same size as the input image) may be generated by encoding the click annotations using a map, such as a disk map or Euclidean distance map. Disk maps, for example, can be generated by starting with an image of value zeros, changing pixel values to 1 (or another positive value) in the clicked pixels and then changing the values of pixels surrounding these clicked ones to 1 to expand the click neighborhood into disk-shaped image regions of value 1. Click maps can be generated by setting values of the clicked pixels (and only the clicked pixels) to be different than the rest of the pixels. Square maps can be generated to include square-shaped regions surrounding clicked pixel locations. In some instances, a single map indicates positions of clicks of multiple classes. In some instances, a separate map is generated for each class.

The masks can be used together with the input image as model training and/or inference input to inform the model where the user input are, so that the model can generate segmentation masks according to where the users click. In training, the model can be trained how to respond to any objects in an image that users would like to target and provide click input for. In inference, the model can predict a target image region/object according to where the user clicks (there can be many objects in an image and the model can use the click locations to predict which object/image region the user intends to segment).

These encodings are further detailed in K. Sofiiuk, I. Petrov, O. Barinova, A. Konushin, F-BRS: Rethinking backpropagating refinement for interactive segmentation, in: 2020 IEEE/CVF Conference on Computer Vision and Pattern Recognition (CVPR), IEEE, 2020, pp. 8623-8632. doi:10.1109/cvpr42600.2020.00865 and in Sofiiuk, Konstantin, Ilia A. Petrov, and Anton Konushin. “Reviving Iterative Training with Mask Guidance for Interactive Segmentation.” arXiv preprint arXiv:2102.06583 (2021), each of which is hereby incorporated by reference in its entirety for all purposes.

Because the classes in the training data need not have semantic meaning, the deep learning model trained on the training data can be configured to provide segmentation in a manner separate from region labeling. Thus, the deep learning model may be configured to predict which portions of an image correspond to different things without predicting what type of thing is depicted in a given portion and/or without predicting whether multiple portions depict the same type of thing. Because of this, a multi-class training data set may be used that includes images and annotations from multiple domains.

Domains may include, for example, digital pathology datasets, natural-scene image datasets, immunohistochemistry datasets, H&E datasets, or any other appropriate image-annotation pair dataset as would be understood by one skilled in the art. Using training data from one or more domains other than digital pathology (e.g., potentially in addition to training data from the digital-pathology domain), a larger training set can be collected. However, by continuing to include digital pathology images in the multi-class training data set, the deep learning model may be readily applied to process multiple types of digital pathology images without substantial additional model development. In some instances, however, the training data set does not even include training data from a domain in which the trained deep learning model is later used for image annotation.

### Deep-Learning Model and Training

The deep learning model can include a neural network with more than three, more than four, or more than five layers (including the input and output layers). The deep learning model can include a convolutional neural network.

The deep learning model can be trained using the training data described here (e.g., that includes multi-class images and that may include ground-truth masks and/or click annotations), such that it learns how to segment a particular number of classes (e.g., two target classes and one background class). The training data enforce network learning of effective representations to match the segmentation prediction that correspond to specific pixels indicated via click annotations (or to specific regions within a ground-truth mask). For example, such a model learns to segment any targeting regions pointed to by these pixel locations. It is thus trained to group together the image pixels of unified labels (i.e., similar/identical network representations) as the “annotated” image pixels, no matter what exact underlying semantic meaning they have. In other words, this model need not learn to differentiate the specific semantic classes in an image, but rather can learn to identify semantically similar image regions with the “annotated” pixels and only group these pixels together to generate a segmented target region. Due to such a design, the interactive deep-learning model is capable of identifying any target object or region a user provides click annotations for and does not require domain-specific training to identify the exact classes of regions. Therefore, such embodiments provided herein pose little restrictions on whether test image and training image come from a same domain. This feature is the key to the powerful capability to generalize across image domains.

Accordingly, the trained deep learning model can be used to process other images to generate predicted segmentation annotations.

In some instances, a user may provide click annotations with an input image. For example, the user may select or upload an image using a GUI. The GUI may then present the image and avail tools to add click annotations. The click annotations may identify—for each of a set of classes—one or more pixels in the image that do, or that do not, correspond to the class. The deep-learning model may use the click annotations and the learned model parameters to predict which portions of the images correspond to each of the set of classes.

In some instances, a user provides an input image but no click annotations. The deep-learning model may then use the learned model parameters to identify a set of segments. The model may attempt to assign labels based on similarities across various regions or may provide merely the segmentation data (e.g., identifying pixels on boundaries of various segmentation regions).

In some instances, an output of a trained deep learning model may include one or more values for each pixel. The value(s) may include a class assignment (e.g., Class 1, 2, or 3). In some instances, a value may include an error indicating that a class was not assigned. The output may include a set of masks—each of which correspond to a given predefined class. Each mask may include a value for each pixel, where a particular value (e.g., 1) indicates that it is predicted that the pixel is part of a region (or a perimeter of the region) of the class and another particular value (e.g., 0) indicates that it is predicted that the pixel is not part of a region (or a perimeter of the region) of the class. Alternatively, a value for each pixel may represent a predicted likelihood or confidence that the pixel is part of a region of the class.

### Interactive GUI for Efficient Label Identification

After the deep learning model processes an input image, a GUI can be generated or a GUI (e.g., a same GUI as used to provide click annotations) can be updated to represent segmentations generated via the processing.

Specifically, the GUI can be configured to receive corrections for any labels and/or to identify—for each of one or more regions—a region label that is to be assigned to the region. For example, input-translation definitions may be created that indicates—for each of multiple particular types of mouse clicks—what the updated region label is to be. To illustrate, a single left click may correspond to a tumor region; a single right click may correspond to a stroma region; and a double left click may correspond to a background region. If a user then performs any of these types of clicks within a depiction of a given segmented region, the region label of the region may be updated or defined accordingly.

As another example, the GUI may be configured such that a pull-down menu (identifying different potential region labels) appears after a user clicks within a given region. The pull-down menu may identify the potential region labels, and the user may select which region label is to be assigned to the region.

In this manner, regions can be quickly identified and classified very quickly in a manner that requires little time invested by the user (e.g., as compared to traditional manual segmenting and manually identifying a region label for each region). For example, an image tile of 600×600 pixels in size can be segmented in less than a minute, and the GUI can facilitate rapid identification of region labels, which can result in real-time updates to region labels and updates to the GUI that are intuitive to users and facilitate further region-label identifications.

The label corrections may quickly or immediately trigger an update of the labels.

### Exemplary Practical Applications

The region labels may be used (for example) to generate one or more metrics or recommendations to support a care provider determining a diagnosis, prognosis or treatment recommendation for a subject. An exemplary metric may include an absolute cumulative area of each of one or more types of regions (e.g., tumor regions and/or stroma regions) in one or more digital pathology images and/or a relative cumulative area of tumor regions in one or more digital pathology images (e.g., relative to a sum of the areas of the tumor and stroma regions). In some instances, a change metric is generated that indicates an extent to which a cumulative absolute or cumulative relative area has changed relative to a comparable metric generated by processing digital pathology images corresponding to a same subject but a prior time point. The change metric may identify a difference or a percentage change or consist of a loss function of the typical kind used in optimization and artificial neural networks.

The region labels may further or alternatively be used (for example) to facilitate performing digital pathology analyses that include characterizing cells depicted in the images. For example, background colors representing the region labels may be provided in a digital-pathology interface presented to facilitate segmenting and/or labeling individual cells. As another example, the region labels may be used to assess one or more rules that determine whether there is a potential inconsistency (or an actual inconsistency) with a cell label and a region label. If so, an alert may be presented or sent to request review of the inconsistency.

### Exemplary Advantages

Thus, embodiments disclosed herein provide a platform that supports simultaneous segmentation of more than two types of regions and that shows success in DL-based interaction segmentation being applied across distinct imaging modalities. Techniques disclosed herein have distinct performance advantages over alternative approaches.

For example, active learning uses heuristic scoring strategies to query a small subset of unlabeled examples (which are the most informative within the dataset), and a model is retrained by iteratively adding only a small set of such set selected examples. While this avoids the need to annotate many images, models developed with active learning only apply to test images of a same or identical distribution as the training images. Thus, active learning cannot be used to produce a generalized model suited for the high variability in dyes, scanners, tissues, etc. that exist in digital pathology.

As another example, techniques disclosed herein provide a GUI that is configured to allow a user to very efficiently provide labeling input. While other annotation tools have been developed based on machine learning or deep features, such approaches require pre-trained models on a similar segmentation task to the input images, which significantly limits model generalizability. Further, such approaches are limited to binary segmentation, whereas most applications in digital pathology require simultaneous identification of three or more different types of target regions.

As yet another example, while transfer learning and domain adaptation improve model generalization to a certain extent, they require additional model training with additional labeled examples from the image domain of interest. Although unsupervised domain adaptation aims to use no annotations from the target domain, it still relies on a validation set from the targeting domain for model selection, which inevitably tends to overfit to the validation set. In addition, a prerequisite for these approaches to work well is the existence of similarities between the image domains. Therefore, such approaches provide neither real-time prediction modification, nor cross-domain generalizability without assumptions about image domains or without additional model retraining.

Additionally, many existing segmentation models (both optimization-based and learning-based approaches) are designed to segment individual objects one at a time, which simultaneously identify two different types of regions with binary segmentation: a foreground object and non-target background. A foreground object (“foreground” for short) is defined as a target object instance in an image, while non-target background is anything else besides the target object. Meanwhile embodiments of the present invention result in segmentation of target objects, no matter if it is a single connected region or multiple disconnected regions of a same class.

### Exemplary Network for Digital Pathology Image Generation and Processing

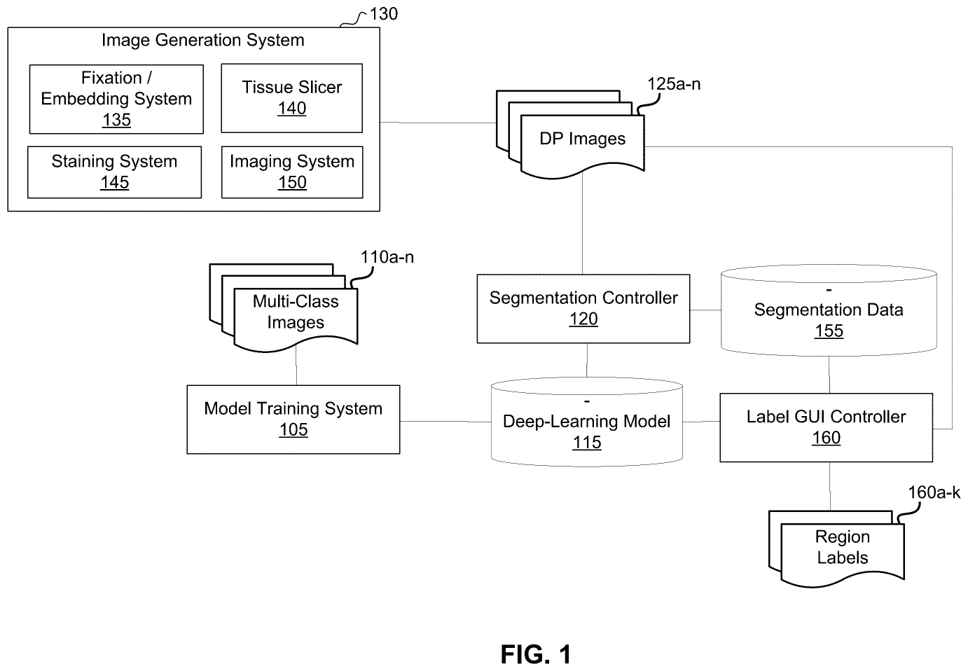

FIG. 1 shows an exemplary network for generating a digital pathology image and labeled regions of interest. It will be appreciated that each of one or more of the depicted components in FIG. 1 (e.g., model training system 105, segmentation controller 120, imaging system 150, label GUI controller 160, etc.) may include one or more processors and a computer-readable medium with instructions that, when executed, perform actions identified herein. In some instances, a single physical or virtual computing system includes one or more processors and a computer readable medium with instructions that, when executed, perform actions described as being performed by multiple components in FIG. 1. For example, a single computing system may perform actions described herein as being performed by model training system 105, segmentation controller 120, and label GUI controller 160.

The network includes a model training system 105 that retrieves or retrieves multi-class images 110a-n from one or more data sources. The one or more data sources may include one or more remote data sources and/or one or more local data sources. In some instances, at least some of the multi-class images 110a-n are retrieved by sending an online request for images and downloading images. Each of the multi-class images 110a-n can include annotation data that indicates borders of or areas of depictions of different segments in the image. The segments in each of the multi-class images 110a-n may correspond to different types of objects or things. For example, rather than only identifying where a vehicle is depicted in an image, the annotation data may identify depictions of each vehicle, each stop light, each crosswalk, each person, etc.

The multi-class images 110a-n may, but need not, be from different domains. The multi-class images 110a-n may include (for example) one or more natural-scenes images, one or more digital pathology images, one or more immunohistochemistry images, one or more H&E images, one or more MRI images, and/or images from one or more other domains. The multi-class images 110a-n may, but need not, include one or more digital pathology images. The multi-class images 110a-n may include one or more images that are not digital pathology images.

Each of the multi-class images 110a-n may be accompanied by segmentation annotation data. The segmentation annotation data may include annotations as disclosed herein. For example, the segmentation annotation data may include one or more click annotations, each of which correspond to a specific pixel in the image and a specific positive label (e.g., identifying a particular class) of negative label. As another example, the segmentation annotation data map include a transformed version of one or more click annotations (e.g., where each click annotation was transformed using a map, such as a click map or Euclidean map). As another example, the segmentation annotation data may include one or more ground-truth masks that indicate—for each pixel—an identification or prediction as to whether the pixel corresponds to a particular label. The segmentation annotation data may be identified by or generated based on input from a user interacting with a GUI that shows each training image. It will be appreciated that a user providing input for the segmentation annotation data need not be the same user as one providing a subsequent image (and/or other related information) to be processed by the trained model.

The model training system 105 may use the multi-class images 110a-n (e.g., and any accompanying segmentation annotation data) to train a deep-learning model 115. The deep-learning model 115 may include a neural network with more than three, more than four, more than five, or more than six layers. The deep-learning model 115 may have a convolutional neural network architecture. Training the deep-learning model 115 may include learning the values of a set of learnable parameters (e.g., weights assigned to connections between various nodes) of the model.

After the deep-learning model is trained, a segmentation controller 120 can use the trained deep-learning model to process each of one or more digital-pathology (DP) images 125a-n. Each DP image 125 may have been generated by an image generation system 130, which may be remote from or local with segmentation controller 120. It will be appreciated that some actions performed by image generation system 130 (and/or by one or more components thereof) are performed automatically, while others (in some instances) are performed using at least some human action.

As part of the image generation system 130, a fixation/embedding system 135 fixes and/or embeds a tissue sample (e.g., a sample including at least part of at least one tumor) using a fixation agent (e.g., a liquid fixing agent, such as a formaldehyde solution) and/or an embedding substance (e.g., a histological wax, such as a paraffin wax and/or one or more resins, such as styrene or polyethylene). Each slice may be fixed by exposing the slice to a fixating agent for a predefined period of time (e.g., at least 3 hours) and by then dehydrating the slice (e.g., via exposure to an ethanol solution and/or a clearing intermediate agent). The embedding substance can infiltrate the slice when it is in liquid state (e.g., when heated).

A tissue slicer 140 then slices the fixed and/or embedded tissue sample (e.g., a sample of a tumor) to obtain a series of sections, with each section having a thickness of, for example, 4-5 microns. Such sectioning can be performed by first chilling the sample and the slicing the sample in a warm water bath. The tissue can be sliced using (for example) using a vibratome or compress to me.

Because the tissue sections and the cells within them are virtually transparent, preparation of the slides typically includes staining (e.g., automatically staining) the tissue sections in order to render relevant structures more visible. In some instances, the staining is performed manually. In some instances, the staining is performed semi-automatically or automatically using a staining system 145.

The staining can include exposing an individual section of the tissue to one or more different stains (e.g., consecutively or concurrently) to express different characteristics of the tissue. For example, each section may be exposed to a predefined volume of a staining agent for a predefined period of time.

One exemplary type of tissue staining is histochemical staining, which uses one or more chemical dyes (e.g., acidic dyes, basic dyes) to stain tissue structures. Histochemical staining may be used to indicate general aspects of tissue morphology and/or cell microanatomy (e.g., to distinguish cell nuclei from cytoplasm, to indicate lipid droplets, etc.). One example of a histochemical stain is hematoxylin and eosin (H&E). Other examples of histochemical stains include trichrome stains (e.g., Masson's Trichrome), Periodic Acid-Schiff (PAS), silver stains, and iron stains. The molecular weight of a histochemical staining reagent (e.g., dye) is typically about 500 kilodaltons (kD) or less, although some histochemical staining reagents (e.g., Alcian Blue, phosphomolybdic acid (PMA)) may have molecular weights of up to two or three thousand kD. One case of a high-molecular-weight histochemical staining reagent is alpha-amylase (about 55 kD), which may be used to indicate glycogen.

Another type of tissue staining is immunohistochemistry (IHC, also called “immunostaining”), which uses a primary antibody that binds specifically to the target antigen of interest (also called a biomarker). IHC may be direct or indirect. In direct IHC, the primary antibody is directly conjugated to a label (e.g., a chromophore or fluorophore). In indirect IHC, the primary antibody is first bound to the target antigen, and then a secondary antibody that is conjugated with a label (e.g., a chromophore or fluorophore) is bound to the primary antibody. The molecular weights of IHC reagents are much higher than those of histochemical staining reagents, as the antibodies have molecular weights of about 150 kD or more.

The sections may then be individually mounted on corresponding slides, which an imaging system 150 can then scan or image to generate raw digital-pathology images 125a-n. Each section may be mounted on a slide, which is then scanned to create a digital image that may be subsequently examined by digital pathology image analysis and/or interpreted by a human pathologist (e.g., using image viewer software).

A label GUI controller 160 can generate, configure or update a GUI to present a given digital-pathology image that is associated with a user. The given digital pathology image may be one queued for segmentation and label processing or one that has been identified in a request for segmentation and label processing. The GUI may be configured to receive one or more annotations. For example, the GUI may include input components that allow a user to select a set of pixels in the digital pathology image and to indicate—for each of the set of pixels—a label that is (or is not) to be assigned to the pixel (e.g., a first type of region, a second type of region or background).

The segmentation controller 120 can process each digital-pathology image 125a-n and any annotations using the trained deep-learning model 115 to generate segmentation data 155. The segmentation data identifies—for each segment detected by the deep-learning model 115—where the segment is within the corresponding digital-pathology image 125. For example, for a given segmented region, the segmentation data 155 may identify pixels that are in a border of the region and/or pixels that are in the segmented region. In some instances, segmentation data 155 includes an annotation image that indicates—for each of one or more pixels—whether the pixel has been assigned to a type of region or a segmented region and, if so, which one. For example, the annotation image may indicate to which of a predefined set of classes (e.g., a first type of region, a second type of region, or background) a pixel has been assigned. Though the classes may lack semantic meaning, the training may be performed such that any class assignment is likely to be consistent with any annotations provided with the respective digital pathology images 125a-n. As another example, each segmented region may be assigned an identifier that is unique relative to other segmented regions in the image. For each segmented region, the pixels that are within the segmented region (or, alternatively or additionally, are part of a boundary of the segmented region) can be assigned a value equal to the unique identifier.

A label GUI controller 160 can generate, configure or update a GUI to present a given DP image (e.g., at a reduced intensity) and to show the boundaries of each segmented portion. When the deep-learning model is configured to assign labels, the labels may also be represented (e.g., via a shading, color of a background, color of a symbol, etc.)

The GUI can also be configured to receive input that identifies or updates a region label for each region. The GUI may be configured to allow a user to zoom in and out of the image and/or to move the field of view. This can allow the user to better see details, such as the morphologies and sizes of cells within a given segmented region.

The GUI may be configured such that, for each segmented region, a user can select from among a predefined set of region labels. When label data was provided as part of the training data, the deep-learning model may have generated a predicted class for each region. In that case, an indication of the predicted class may be presented for each segmented region (e.g., via a shading, background color, symbol color, symbol shape, etc.). When label data was not provided as part of the training data (or when insufficient label data was provided), all regions may be represented as having an indication that a region label has yet to be assigned, though a region label may be subsequently assigned to each segmented region (and the representation may be updated accordingly) based on label-selection input provided by a user. In other instances, an initial region label is automatically assigned to each region, and a user may selectively provide input to change the label when the user believes that the initial label was incorrect. In this case, all regions may be assigned a same initial region label (e.g., a label that is statistically most common), or one or more rules may be evaluated to determine which initial region label to assign to each region (e.g., based on an area of the region, a perimeter of the region, a perimeter-to-area ratio of the region, a number of cells detected within the region, etc.).

In any case, the GUI may be configured to receive input that identifies (e.g., as an initial indication or as an update indication) a label that is to be assigned to one or more portions of the image. For example, a user may click on a marker that is color coded to use a radio-button list to identify an initial labeling and may then provide input that identifies a different label. As another example, a user may click within a segmentation region depiction and may then use a defined mouse click signature so as to identify an initial (e.g., and final) label) for the region.

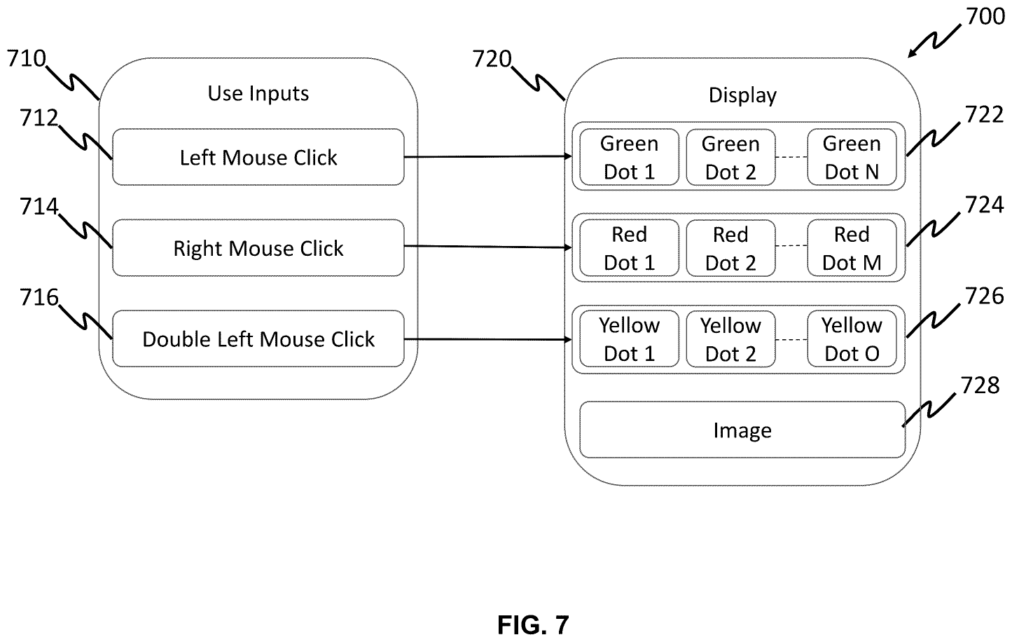

In some instances, a distinct type of mouse click and/or touchscreen interaction may be assigned to each of the predefined set of region labels. For example, each of: (1) a single left mouse click; (2) a single right mouse click; and (3) a double left mouse click can be assigned to one of the region-label options. (FIG. 7 and the associated descriptions provide an illustration of a mapping of mouse clicks to labels.) As another example, each of (1) short hold and then swipe down slightly; (2) short hold and then swipe up slightly; and (3) short hold and then swipe left slightly may be assigned to one of the region-label options.

The GUI may also include an option to (for example) assign any and all currently unlabeled segmentation regions in an image or all currently unlabeled segmentation regions fully in a field of view to a given region label (which may be selected by the user). Thus, for example, within a given field of view, the user may then choose to label all stroma and background regions and to then indicate that all other regions are to be labeled as tumor regions. The GUI may alternatively or additionally be configured such that a user can select a tool to define a rectangular area in a depicted image and then to assign all regions within the area to a selected region label.

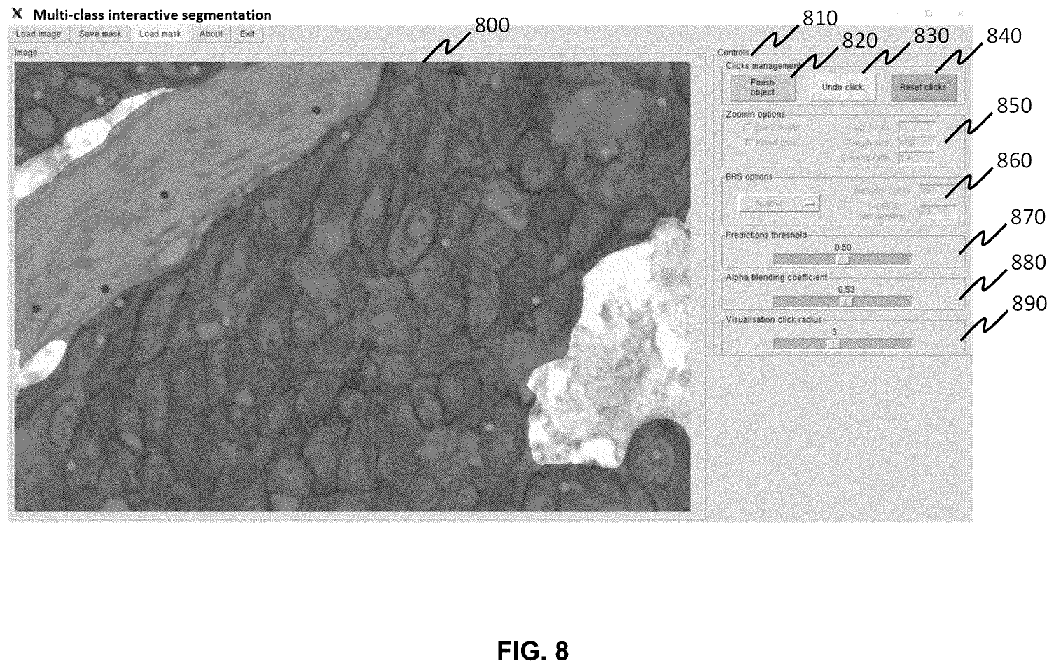

As each label is assigned or updated, the GUI may be updated to reflect the label (e.g., by adding a background color, which may represent the assigned label, to the region), such that the user can easily see the current labels (and, if applicable, which regions remain unlabeled). The GUI may include a submit or completion input option that can be selected by the user when the user is satisfied with the labels. Further details of the GUI are provided herein, such as when describing FIG. 8.

Label GUI controller 160 may store and/or output region region labels 160a-k. The region labels 160a-k may (for example) include a mapping that associates each unique region identifier with a region label (or region-label identifier). The region labels 160a-k may alternatively or additionally include a mapping that associates, for each of some or all pixels in the image, the pixel with a region label (or region-label identifier).

### Exemplary Interactive Segmentation Workflows

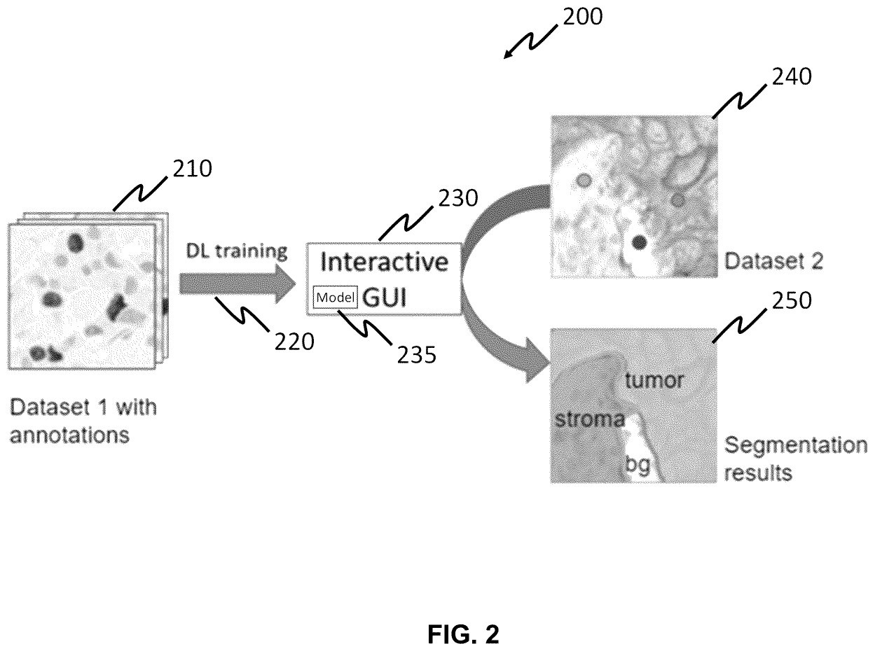

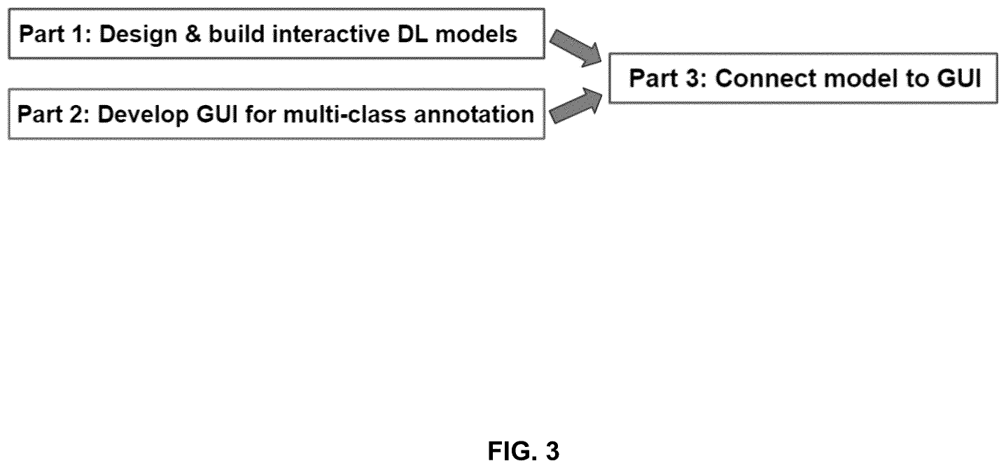

Referring to FIG. 2, an embodiment of a multi-class interactive segmentation workflow 200 is depicted. Various actions in the multi-class interaction segmentation workflow 200 may be performed by model training system 105, segmentation controller 120 and/or label GUI controller 160. FIG. 3 depicts an example of a corresponding pipeline for model building is depicted according to various embodiments.

In various embodiments, the multi-class interaction segmentation workflow 200 designs and builds a deep learning model to perform segmentation on multiple classes and to receive user input that identifies a label for each of one or more pixels and/or segmented region. An interactive GUI may be configured to receive an input image and to potentially initially receive annotation input that identifies a class to which a given point or segment is to be assigned and/or to receive post-training input that identifies or updates a label to be assigned to a given segmented region.

For example, a first dataset 210 may include a plurality of images (e.g., multi-class images 110a-n) from one or more classes (e.g., DP images, natural scene images, immunohistochemistry images, H&E images, or any other relevant images) and may include corresponding annotations (e.g., that identify click annotations, distinct segments within the images, a ground-truth map, etc.). Part or all of the first dataset 210 may have been received via an interactive GUI from a user that is facilitating training a deep-learning model (e.g., deep-learning model 115) that is connected with the interactive GUI.

The plurality of images may include digital pathology images featuring different tissue types and their corresponding segmentation annotations. In other embodiments, the first dataset 210 from another domain, for example, natural scene images may be used. The first dataset 210 may include (for example) annotation data that associates each of one or more pixels or areas with a given class. The class may be one of multiple predefined non-semantic classes.

The deep-learning model may have been designed and built (at part 1 of FIG. 3) by defining the model's architecture, hyperparameters, and loss function. At 220, the first dataset 210 may be utilized (e.g., by model training system 105) to train the deep-learning model. By using datasets from multiple domains, the resulting model that is trained using the data may be more generalizable as compared to using training data from a single domain.

A GUI can also be developed that is configured to display a digital pathology image and segmentation data (e.g., by overlaying segmentation boundaries on the image) and to receive user inputs that identify a label (e.g., of a predefined set of labels) for each section or to correct a predicted label. (See Part 2 of the pipeline of FIG. 3.) This interactive GUI may be, but need not be, a GUI that received part or all of the first dataset. Even if the GUI is the same, the GUI may be configured to present a first interface for receiving part or all of the first dataset to a first user and to present a second interface for receiving label definitions or updates to a second user.

The GUI of part 2 may be connected to the model (see part 3 of the pipeline of FIG. 3). This connection can allow a user to identify (e.g., upload, select, etc.) one or more digital pathology images and/or checkpoint annotation data (e.g., a second dataset of images) and request processing via the GUI. The request can automatically trigger processing by the deep-learning model to identify segmentation, and—once the segmentations are identified—the GUI can automatically display the image and segmentation data and be configured to receive label identifications or updates. In some embodiments, an initial label (e.g., a non-semantic label) may be assigned to each of one or more segmented regions. In some embodiments, at least one segmented region lacks an initial label.

Using an interactive GUI 230 (e.g., controlled by label GUI controller 160), the user may efficiently identify and/or update labels for at least some of the segmented regions. For example, in various embodiments, the user may use a mouse and click on areas to provide annotations. The interface may be configured such that annotations (e.g., identifying new or updated labels) can be made quickly and efficiently and such that the GUI is updated in real-time so as to reflect the most recent labels for the regions. For example, in various embodiments, different colors may be used to annotate different classes. For example, a blue dot may be used to annotate a stroma region, a green dot to annotate a tumor region, and a red dot to annotate a background region. Furthermore, multiple annotations may be used for one or more of the classes.

In various embodiments, the trained deep-learning model 235 is not further modified after the initial training (using the multi-class data). Instead, the deep-learning model 235 outputs segmentations for an image, and the interactive GUI is configured to receive inputs that quickly identify labels for each of some of all of the segmented regions.

In one embodiment, the deep-learning model is used to segment an image tile of 600×600 pixels in less than a minute. Thus, a user may quickly and efficiently annotate an image or perform image segmentation. Further, with no model retraining or fine-tuning required during use, the model does not over-fit to specific datasets and maintains generalizability. In addition, the system provides confidence to users because the process and outputs are not a black-box, such a traditional deep-learning system and instead enables user-guided modifications of model prediction, which provides real-time feedback and thus is interactive, intuitive, and flexible for end-users.

### Exemplary Segmentation Approach Using Limited Annotation

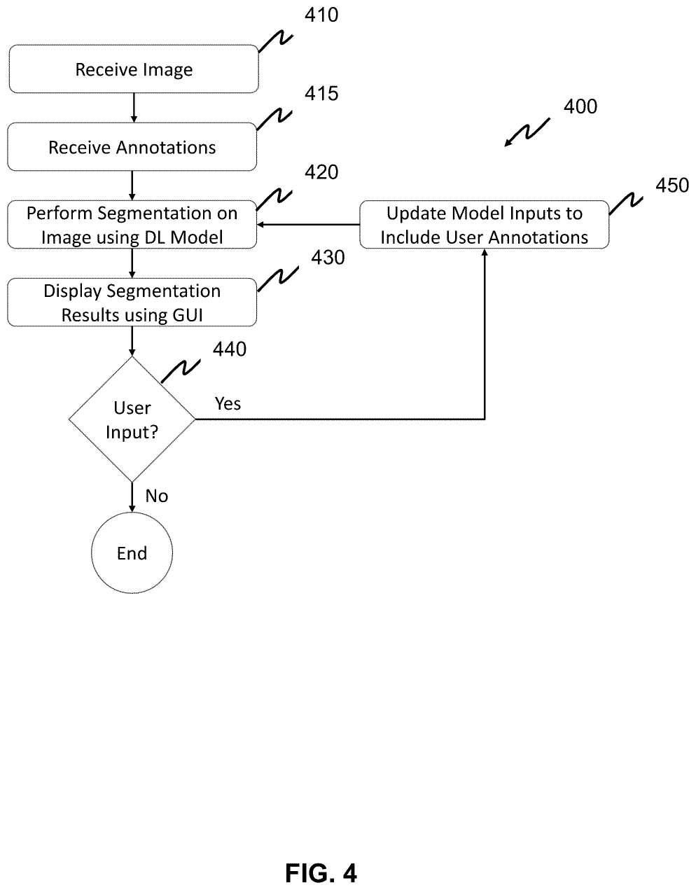

Referring to FIG. 4, an exemplary process 400 of an interactive segmenting is depicted. In various embodiments, the multiclass deep learning system receives an image to be segmented (Step 410). In some embodiments, the image may be a complete image (e.g., a whole slide digital pathology image) or a portion of an image (e.g., a field of view of a complete image, a region of interest of a complete image, a tile of a complete image, etc.). In various embodiments, the user may provide one or more annotations for the image (Step 415). Such annotations may include (for example) click annotations, which may potentially be further processed to be transformed to other annotations using a transformation algorithm. In various other embodiments, no annotations may be provided by the user before the initial segmentation.

Segmentation may be performed on the image (and its annotations, if available) using the deep learning model (Step 420). In various embodiments, the model may be an n-class model. Thus, the model may be configured to segment the input image in up to n types of different areas in each round of user interaction, which generates a single segmentation mask with up to n classes of areas. For example, in various embodiments, the deep learning model is a three-class model configured to classify the input image into areas including tumor cells, stroma cells, and background tissue. In other embodiments, the deep learning model is a four-class model configured to classify the input image into four different areas including immune cells, tumor cells, stroma cells, and background cells. It should be understood by those skilled in the art that the deep learning model can be configured for any number of classes and to segment any type of tissues or cells.

The deep learning model output provides a segmented image to the interactive GUI and the results are displayed to the user (Step 430). The user may then elect to provide annotations using the interactive GUI (Step 440). When the user provides annotations, the interactive GUI provides the annotations to the deep learning model as additional inputs (Step 450) and the process may be repeated as many times as are required to achieve satisfactory results.

### EXAMPLES

### Example 1

Traditional interactive segmentation models, both optimization-based and learning-based algorithms, are designed to segment individual objects one at a time, which simultaneously identify two different types of regions with binary segmentation: a foreground object and non-target background. A foreground object (“foreground” for short) is defined as a target object instance in an image, while non-target background is anything else besides the target object.

Techniques disclosed herein can generate strong results where the targeting objects can be segmented with reasonable success, no matter if it is a single connected region or multiple disconnected regions of a same class. An interactive binary segmentation was used for multi-class segmentation of a DP image.

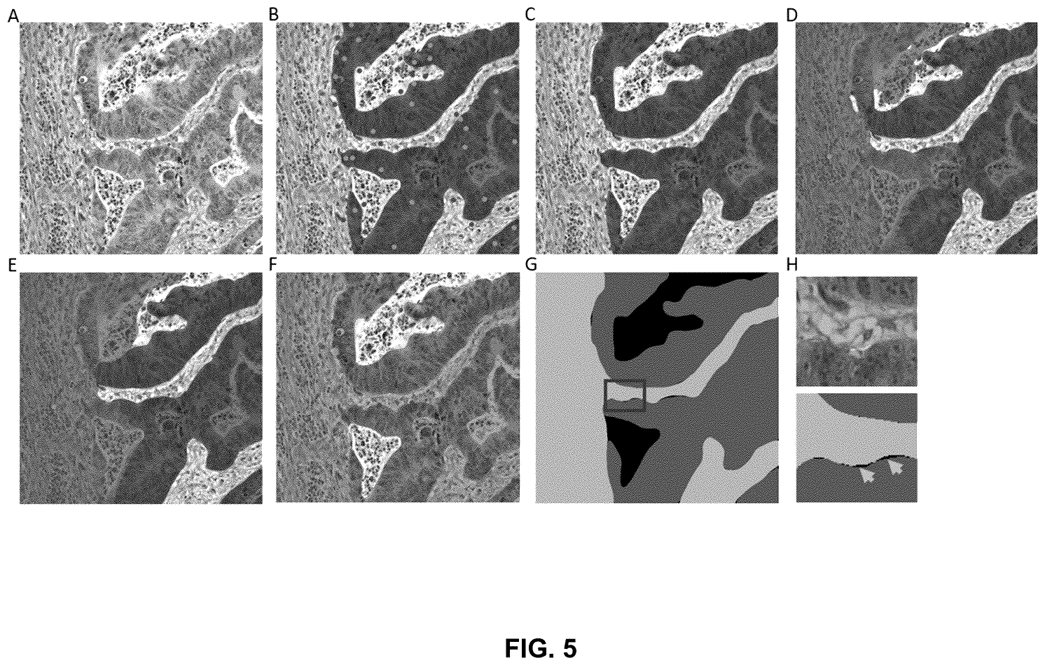

FIGS. 5A-5H illustrate the steps performed when binary segmentation is used to segment a digital pathology image to identify three types of regions. FIG. 5A shows an exemplary colon cancer H&E image tile. FIG. 5B shows a first round of binary segmentation to identify tumor (red masked regions) and non-tumor (rest of non-masked regions). FIG. 5B depicts the same image tile after a first round of binary segmentation was performed using the deep learning model to identify tumor (red masked regions) and non-tumor (rest of non-masked region). In this example, a user has annotated the image using the interactive GUI 130 with green dots using positive clicks on tumor regions and red dots using negative clicks on non-tumor regions. FIG. 5C shows an exemplary updated tumor region segmentation result (red masked regions). Thus, the tumor regions are identified despite being disconnected in this image.

FIG. 5D depicts the image after a second round of segmentation that identifies stroma and non-stroma regions. At this point, a green dot indicates a first positive click to identify a stromal region. The green masked regions are stroma regions identified in the second round of segmentation after the first positive green click, and the red masked regions are tumor regions identified by the first round of segmentation of tumor versus non-tumor regions. In the depicted example, after the first green click, the prediction of the stromal region extends to the previously identified tumor region, due to the second round of segmentation is probably independent of the first round.

FIG. 5E depicts an example of correcting stroma segmentation results where a red dot (negative click) has been added. FIG. 5F depicts a series of click annotations that modify the second round of segmentation. FIG. 5G depicts the final segmentation result for tumor, stroma and background regions where light blue corresponds to stroma regions, gray corresponds to tumor regions, and black corresponds to background regions. FIG. 5H depicts zoomed-in views from the red rectangle region from FIG. 5G. The upper image includes segmentation results overlaid on the original image and the lower image shows the segmentation mask that was used. In this example, the arrows point to gaps after combining masks generated from two rounds of segmentation.

Since each round of binary segmentation is independent, users need to make corrections for the already segmented objects from previous rounds (FIGS. 5D-5E). This prolongs the analysis time because of the corrections needed for the previously identified regions in addition to the targeting classes in the current round of segmentation (FIG. 5F). Such corrections to remedy the already annotated regions is redundant and time-consuming. In addition, users need to carefully provide click annotations via clicks at the boundaries of the targeting classes, otherwise gaps often occur between segmented objects (FIGS. 5G-H) from different rounds of segmentation and thus inevitably leading to confusions and errors.

### Example 2

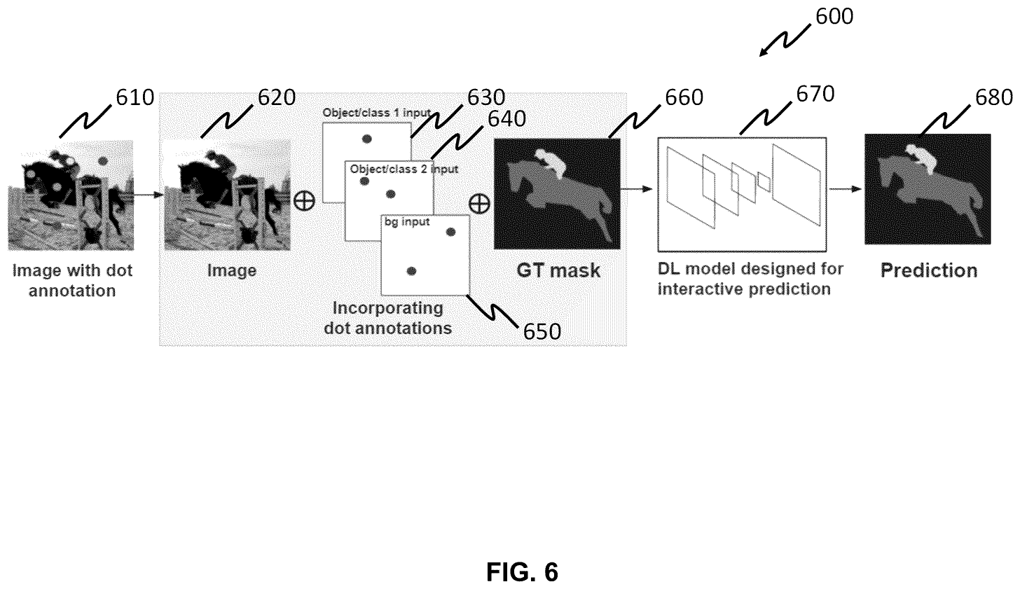

FIG. 6 shows an exemplary representation of the use of a three-class deep learning model 600. For a three-class model, the ground truth masks contain up to three different types of regions, and user annotations contain up to three sets of user annotations, with each corresponding to one type of region.

The ground truth masks for each segmentation task are instance-level annotations where multiple object instances are annotated. For example, during training, up to two objects are sampled from a mask to generate a new mask, where the rest of the image is regarded as non-target background. Such non-target background can contain (a) other objects that are not selected from the original mask, and (b) pure background, where no objects are annotated in the original mask.

Training data for the three-class deep learning model includes an image with click annotations 610. Colored dots represent the mouse clicks on targeted regions. The location and number of dots are simulated for model training, to mimic user inputs at test time, where a user provides mouse clicks to guide the segmentation of targeting regions.

Simulated click annotations are used during training. For example, mouse clicks annotations are encoded into disk maps or Euclidean distance maps, which form additional masks that can be conveniently fed into a deep learning model together with a training image. Various strategies of click simulation may be utilized for binary interactive segmentation, including positive click sampling and negative click sampling. Positive click sampling mimics users' behaviors of mouse clicking on a target object by sampling a variable number of pixel locations randomly within the target object region in the mask. Negative click sampling mimics users' behaviors for identifying background (i.e., anywhere not on the target object). In various embodiments, three strategies of negative click sampling may be used. For example, a first strategy may include using a variable number of pixel locations randomly sampled from the pure background regions. A second strategy may include using a variable number of pixel locations are sampled from unselected objects in the ground-truth masks. A third strategy may include using a variable number of pixel locations from the pure background are sampled close to the boundaries of the target object.

In this Example, to train a three-class deep learning model, negative click sampling is used for one type of region and positive click sampling for the other two types of regions. It will be appreciated that negative clicks may be better for effectively correcting erroneously segmented regions. Thus, keeping such negative click sampling for one of the classes provides the flexibility for users to perform negative clicks for the class of region that is most difficult to segment.

The image with annotations 610 is split into an image 620 and annotations for each object/class being segmented by the deep learning model 600. For example, the three-class deep learning model includes a first class annotation input 630, a second class annotation input 640, and a background annotation input 650. The image 620 and the annotations 630, 640, 650 are then used to generate a ground truth mask 660, which is used to train the deep learning model 670 which can then generate the prediction 680.

### Example 3

FIG. 7 depicts an example workflow of providing annotations via an interactive GUI configured to provide user-input functionality for multiple segmentation classes. Annotations are provided by the user using various user inputs 710 and the GUI is generated on a display 720. The user inputs 710 may include any computer input device such as a mouse, a touch screen, a keyboard or other input device. The user inputs 710 include a mouse with at least two buttons that include a left click button and a right click button.

In a typical binary object segmentation GUI, left mouse clicks are used as positive clicks (i.e., a location is part of a class) and right mouse click as negative clicks (i.e., a location is not part of a class). Using a multi-class interactive GUI, left and right click buttons may be mapped to a class. For example, in a three class system, a left mouse click 712 may be associated with a first class, a right mouse click 714 may be associated with a second class, and a combination mouse click 716 may be associated with a third class. Each of the mouse clicks may cause the interactive GUI to overlay a dot corresponding to the class associated with the click onto the image 728.

A left mouse click 712 may be associated with a first type of region (e.g., a tumor region), and each left mouse click triggers the display of a green dot 722 on the interactive GUI via the display 720. The left mouse click 712 may serve as a positive click. A right mouse click 714 may be associated with a second type of region (e.g., a stroma region) and each right mouse click triggers the display of a red dot 724 by the GUI, and may also serve as a positive click. A double-left mouse click 716 may be associated with a third type of region and may produce a yellow dot 726. In various embodiments, the double-left mouse click 716 may serve as a negative click. To connect a trained model to the interactive GUI, the GUI backend is configured to convert the user clicked pixel locations for three classes to three disk maps and to use these disk maps along with the image as deep learning model inputs for generating predictions for the corresponding three-classes.

Using the interactive GUI, a user may initiate segmentation by adding a first positive click on a target region of interest. This click triggers an initial network prediction. Adding additional clicks triggers the changes of network input, which leads to corresponding changes in the segmentation prediction. The segmentation results can be displayed as a colored overlay on the input image.

### Example 4

FIG. 8 depicts an example interactive GUI for defining click annotations. The interactive GUI includes an image display area 800 and a control panel 810. The image display area 800 is configured to show the image being segmented and provides an area for users to add annotations. The controls allow for a variety of options for operating the system. For example, the interactive GUI may include buttons to finish 820, undo click 830, and reset clicks 840. The GUI controls 810 may also include various zoom options 850. In various embodiments, the user may be able to use the GUI controls to adjust various model parameters. For example, the GUI controls 810 may include back propagation refinement (BRS) options 860, and sliders to modify a prediction threshold 870 and alpha blending coefficient 880. Other control options include a slider for dot radius 890.

### Example 5

A title by tile approach is used for whole-slide image segmentation. Tile by tile prediction for WSI. With a tile-by-tile approach, a user performs interactive segmentation one image tile at a time. In this way, a user segments one tile to their satisfaction in the manner described above and then moves on to the next. A platform of interest (web application, GUI, etc.) is chosen to display the whole-slide image and support interactive segmentation. Once all tiles are segmented, the platform can then automatically assemble all predictions and display whole-slide segmentation results.

### Example 6

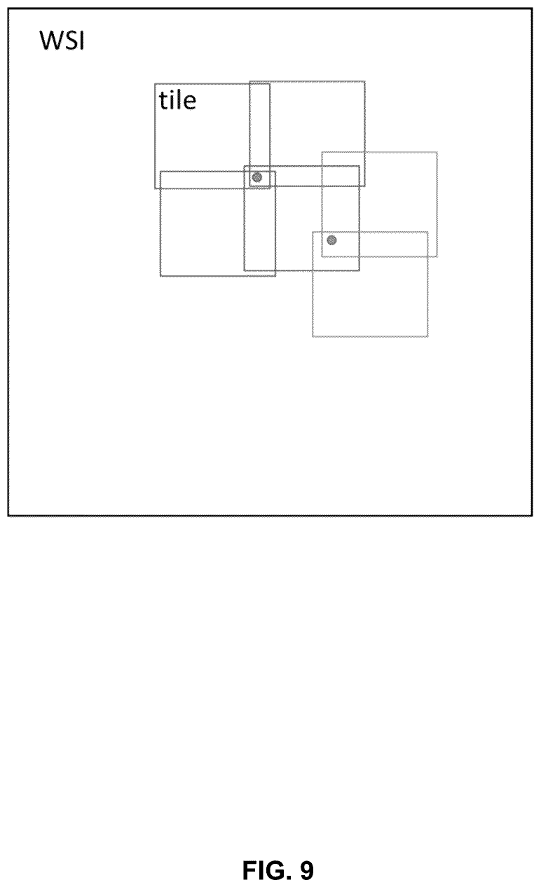

A radial expansion scheme was used for whole-slide image segmentation. FIG. 8 depicts a visualization of a radial expansion strategy for whole-slide interactive segmentation according to various embodiments. The radial expansion approach leverages the benefit of prediction in overlapping tiles to expand prediction in a radial manner and thus expedite segmentation in a large image like WSI (FIG. 9). For example, when a user starts analysis by clicking anywhere in a WSI (for example, the blue dot in FIG. 9), multiple tiles that cover this click are selected to maximize the total tile coverage, so that predictions for all these tiles can be generated (blue tiles in FIG. 9). In the next step, a user can click anywhere in predicted regions that need to be corrected (example second click shown as orange dot in FIG. 9). The correcting clicks help expand prediction to tiles that cover these clicks (orange tiles in FIG. 9). Since one user click triggers segmentation in multiple tiles, performing such prediction with the designed radial expansion scheme enables much faster propagation of segmented regions in the whole-slide imaging, and thus expedited whole-slide image segmentation. As another advantage of this approach, the system can leverage parallelized or distributed model inference of multiple tiles to further expedite whole-slide image segmentation.

### Example 7

Domain-specific and dataset-specific whole-slide image segmentation is provided. In use cases where segmentation is needed for a specific dataset with multiple whole-slide images, interactive segmentation can serve both as an initial annotation-generation strategy and fast annotation expansion strategy. For example, first, a subset of slide image regions can be selected either manually, randomly, or via active-learning-based approaches. Then, segmentation masks of these selected large image regions in selected WSIs can be generated efficiently with one of the aforementioned approaches (i.e., tile by tile prediction or radial expansion strategy), which is then followed by iterative model training and fine-tuning. In this way a model can be quickly built for WSI segmentation of a dataset of interest.

### Example 8

A deep-learning model was trained on the Semantic Boundaries Dataset (SBD), a commonly used benchmark for interactive segmentation with instance-level annotations from the natural scene image domain. SBD contains 11355 images, among which 8498 were used for training and 2857 for validation. In each image, one or multiple foreground objects (i.e. instances) are annotated.

An HRNet with OCR modules was pre-trained on ImageNet as a backbone, whose architecture was recently proposed to enable the identification of fine details in a number of vision tasks, including image segmentation and human pose estimation. To incorporate simulated user clicks into model training, convolutional blocks were separately encoded for images and for disk maps from user inputs. Note that such a backbone can be replaced with another model of choice to accommodate specific use cases. In addition, domain-specific annotated datasets, for example, IHC images with corresponding masks, if available, can be utilized to train such an interactive system.

Implementation details: Three-class click sampling was performed on the fly during training. Specifically, for each image, at most two foreground instances (i.e., individual connected objects in ground-truth masks) were selected randomly from the segmentation mask. Together with pure background annotation, a new mask with at most three different regions was generated. Two of the regions correspond to targeting objects, and one corresponds tonon-target background. From such a new mask, positive clicks were randomly placed inside each foreground object region, while negative clicks were placed in the background regions with the same strategies. Images were cropped into 320×480 patches for training and augmented with random rescaling, flipping and color jittering.

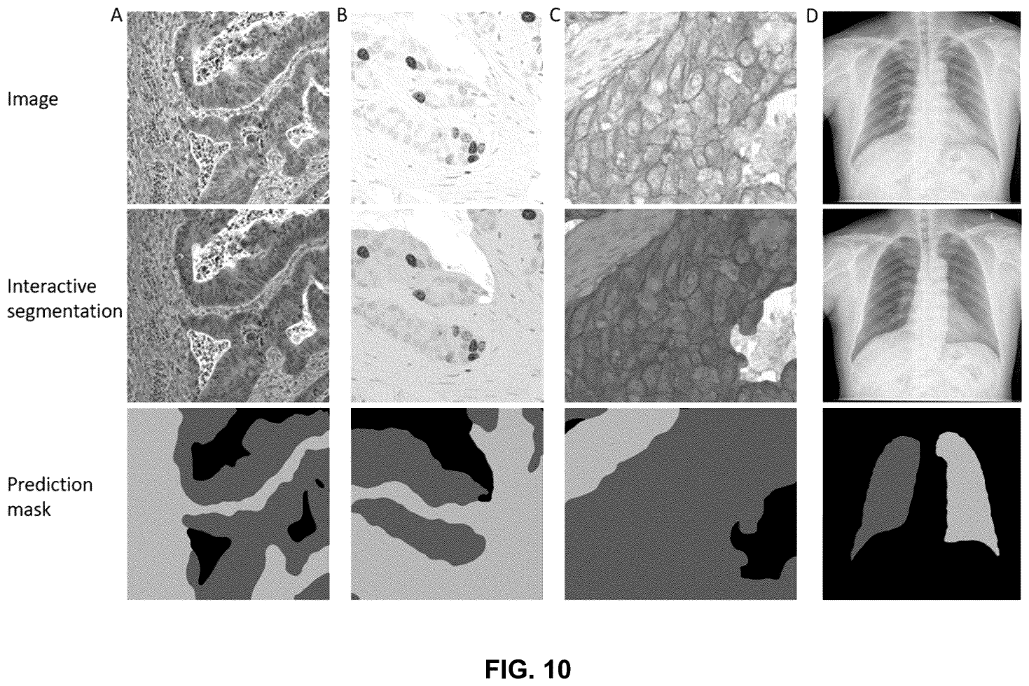

The interactive platform was used to segment images of four different classes, as shown in FIG. 10. Specifically, FIG. 10 depicts images that include (A) Model performance on an example colon cancer H&E image tile (B) Model performance on an example IHC image from breast cancer tissue targeting Ki67 (brown DAB stain) and counterstained with hematoxylin (blue stain), (C) an example IHC image from lung cancer tissue targeting PDL1 (pink Tambra stain) and CK7 (yellow Dabsyl stain) with hematoxylin counterstain (blue), and (D) an example chest X-ray image. In the bottom panel of A-C, light blue regions are stroma, gray regions are tumors and black regions are non-target background. In the bottom panel of D, the light blue region is the right lung, the gray region is the left lung and the black region is non-target background. The predictions demonstrate that the proposed model design and training strategies successfully segments images for three classes of regions interactively, as demonstrated in FIG. 10.

Some embodiments of the present disclosure include a system including one or more data processors. In some embodiments, the system includes a non-transitory computer readable storage medium containing instructions which, when executed on the one or more data processors, cause the one or more data processors to perform part or all of one or more methods and/or part or all of one or more processes disclosed herein. Some embodiments of the present disclosure include a computer-program product tangibly embodied in a non-transitory machine-readable storage medium, including instructions configured to cause one or more data processors to perform part or all of one or more methods and/or part or all of one or more processes disclosed herein.

The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention as claimed has been specifically disclosed by embodiments and optional features, modification, and variation of the concepts herein disclosed may be resorted to by those skilled in the art, and that such modifications and variations are considered to be within the scope of this invention as defined by the appended claims.

The description provides preferred exemplary embodiments only, and is not intended to limit the scope, applicability or configuration of the disclosure. Rather, the description of the preferred exemplary embodiments will provide those skilled in the art with an enabling description for implementing various embodiments. It is understood that various changes may be made in the function and arrangement of elements without departing from the spirit and scope as set forth in the appended claims.

Specific details are given in the following description to provide a thorough understanding of the embodiments. However, it will be understood that the embodiments may be practiced without these specific details. For example, circuits, systems, networks, processes, and other components may be shown as components in block diagram form in order not to obscure the embodiments in unnecessary detail. In other instances, well-known circuits, processes, algorithms, structures, and techniques may be shown without unnecessary detail in order to avoid obscuring the embodiments.

What is claimed is:

1. A computer-implemented method comprising:
receiving an image and displaying the image using a graphical user interface;
receiving at least one first image annotation provided by a user via the graphical user interface;
producing a first segmented image using a deep learning model, wherein the deep learning model uses the digital pathology image and the at least one first image annotation; and
displaying the first segmented image using the graphical user interface;
receiving at least one second image annotation provided by the user via the graphical user interface;
producing a second segmented image using the deep learning model, wherein the deep learning model uses the digital pathology image, the at least one first image annotation, and the at least one second image annotation; and
displaying the second segmented image using the graphical user interface.

2. The method of claim 1, wherein the deep learning model is a three class model and the at least one first image annotation comprises at least one annotation for each class.

3. The method of claim 1, wherein at least one image annotation provided by the user is provided using a mouse click on the displayed image.

4. The method of claim 3, wherein the mouse click comprises a left mouse click associated with a first segmentation class.

5. The method of claim 4, wherein the mouse click comprises a right mouse click associated with a second segmentation class.

6. The method of claim 4, wherein the mouse click comprises a combination mouse click associated with a third segmentation class.

7. The method of claim 6, wherein the combination mouse click comprises a double left mouse click.

8. The method of claim 1, wherein the deep learning model is trained using a first dataset comprising training images with one or more annotations.

9. The method of claim 8, wherein the training images comprise images from a first image domain.

10. The method of claim 9, wherein the training images further comprise images from a second image domain.

11. The method of claim 8, wherein the second image domain is different than the first image domain, and wherein the first image domain and the second image domain each comprise one of: natural scene images, digital pathology images, Immunohistochemistry images, x-ray images, and Hematoxylin and eosin images.

12. The method of claim 8, wherein the one or more annotations comprise one or more simulated click annotations.

13. The method of claim 1, wherein the image is a whole-slide pathology image.

14. A system comprising:
one or more data processors; and
a non-transitory computer readable storage medium containing instructions which, when executed on the one or more data processors, cause the one or more data processors to perform a set of operations including:
receiving an image and displaying the image using a graphical user interface;
receiving at least one first image annotation provided by a user via the graphical user interface;
producing a first segmented image using a deep learning model, wherein the deep learning model uses the digital pathology image and the at least one first image annotation; and
displaying the first segmented image using the graphical user interface;
receiving at least one second image annotation provided by the user via the graphical user interface;
producing a second segmented image using the deep learning model, wherein the deep learning model uses the digital pathology image, the at least one first image annotation, and the at least one second image annotation; and
displaying the second segmented image using the graphical user interface.

15. A computer-program product tangibly embodied in a non-transitory machine-readable storage medium, including instructions configured to cause one or more data processors to perform a set of operations comprising:
receiving an image and displaying the image using a graphical user interface;
receiving at least one first image annotation provided by a user via the graphical user interface;
producing a first segmented image using a deep learning model, wherein the deep learning model uses the digital pathology image and the at least one first image annotation; and
displaying the first segmented image using the graphical user interface;
receiving at least one second image annotation provided by the user via the graphical user interface;
producing a second segmented image using the deep learning model, wherein the deep learning model uses the digital pathology image, the at least one first image annotation, and the at least one second image annotation; and
displaying the second segmented image using the graphical user interface.

16. The computer-program product of claim 15, wherein the deep learning model is a three class model and the at least one first image annotation comprises at least one annotation for each class.

17. The computer-program product of claim 15, wherein at least one image annotation provided by the user is provided using a mouse click on the displayed image.

18. The computer-program product of claim 17, wherein the mouse click comprises a left mouse click associated with a first segmentation class.

19. The computer-program product of claim 18, wherein the mouse click comprises a right mouse click associated with a second segmentation class.

20. The computer-program product of claim 18, wherein the mouse click comprises a combination mouse click associated with a third segmentation class.
